(19) United States
(12) Patent Application Publication
(10) Pub. No.: US 2026/0103665 A1
(43) Pub. Date: Apr. 16, 2026

(54) Synovium-on-a-Chip

(71) Applicant: New York University, New York, NY (US)

(72) Inventors: Weiqiang Chen, New York, NY (US); Chao Ma, New York, NY (US); Benteng Ma, Brooklyn, NY (US); Theresa Wampler Muskardin, New York, NY (US)

(21) Appl. No.: 19/116,645

(22) PCT Filed: Sep. 29, 2023
(86) PCT No.: PCT/US2023/075493

§ 371 Date: Mar. 28, 2025

Related U.S. Application Data
(60) Provisional application No. 63/377,797, filed on Sep. 30, 2022.

(51) Int. Cl. C12M 1/16; C12M 1/00; C12M 1/12; C12M 1/34; C12M 3/00; G01N 33/50

(52) U.S. Cl. CPC C12M 21/16; C12M 23/42; C12M 25/16; C12M 29/10; C12M 41/00; G01N 33/502; G01N 33/5044; G01N 2500/10

(57) ABSTRACT

A synovium on a chip device having: a cartridge housing, a central chamber embedded in the cartridge housing, at least one aperture fluidly connected to the central chamber, and a plurality of evenly spaced micropillars arranged in a substantially circular shape within the central chamber such that the central chamber is partitioned into at least a first inner region and a first outer region, wherein the first inner region includes endothelial cells configured to mimic a vascular network and the first outer region includes fibroblast-like synoviocytes (FLS) configured to mimic a synovium.

### CROSS-PRIORITY TO RELATED APPLICATIONS

This application claims priority to U.S. Provisional Application No. 63/377,797, filed Sep. 30, 2022, the entirety of which is incorporated herein by reference.

### STATEMENT REGARDING FEDERALLY SPONSORED RESEARCH OR DEVELOPMENT

This invention was made with government support under Grant No. R35GM133646 from the National Institute of Health. The government has certain rights in the invention.

### BACKGROUND OF THE INVENTION

In rheumatoid arthritis (RA), delay in effective treatment is associated with increased morbidity and mortality, and a heavy economic burden. Early, effective treatment prevents permanent damage, and remission within the first 3 months of therapy is the best predictor of long-term remission. Unfortunately, current treatment strategies are empiric as there are no markers to determine which therapy is best for an individual, and many patients cycle through ineffective therapies and accrue damage.

Tumor necrosis factor-alpha inhibitors (TNFi) are the most common biologic treatment in RA. Responses are variable, with approximately 30% not responding and 30% responding only partially. Fibroblast-like synoviocyte (FLS) is a major cell type in the synovium, which is reported to release cytokines to drive inflammation. The resulting inflammatory niche will in turn attract immune cells, particularly monocytes, which then exacerbate the tissue inflammation and contribute to joint destruction. Therefore, the role of monocyte and its phenotypes and associated cytokine signaling remained to be elucidated in the RA context.

To study the pathophysiological mechanisms of RA, preclinical studies largely rely on in vitro cell cultures and in vivo animal models. In vitro 2D culture offers a rapid way to analyze disease responses but lacks a 3D tissue environment. In vitro 3D cell culture within a hydrogel can provide the 3D environment, yet it fails to recapitulate the in vivo RA structure. Animal-based studies help improve our understanding of the RA pathogenesis, yet the lack of human immunity compromises the accuracy of obtained results. Recently, Organ-on-Chip is demonstrated to provide a physiologically relevant tissue or disease platform to in vitro interrogate human biology. Thus far, few microfluidic approaches have been successfully applied to reflect the pathophysiology of the RA niche.

Thus, there is the need in the art for an organotypic device to recapitulate the in vivo RA pathophysiology.

### SUMMARY OF THE INVENTION

In some aspects, a synovium on a chip device having a cartridge housing, a central chamber embedded in the cartridge housing, at least one aperture fluidly connected to the central chamber, and a plurality of evenly spaced micropillars arranged in a substantially circular shape within the central chamber such that the central chamber is partitioned into at least a first inner region and a first outer region, wherein the first inner region comprises endothelial cells configured to mimic a vascular network and the first outer region comprises fibroblast-like synoviocytes (FLS) configured to mimic a synovium.

In some embodiments, the first inner region and the first outer region are concentric. In some embodiments, the first outer region comprises a fibrin hydrogel where fibroblast-like synoviocytes (FLS) are embedded to mimic a synovium. In some embodiments, the plurality of micropillars have a cross-sectional shape selected from the group consisting of: circular, ovoid, square, rectangular, triangular, trapezoidal, and polygonal. In some embodiments, the plurality of micropillars is evenly spaced by a distance between about 50 μm and 200 μm.

In some embodiments, the device further comprises one or more sensors comprising capture molecules or probes positioned within the central chamber. In some embodiments, the capture molecule or probe is selected from the group consisting of: antibodies, antibody fragments, antigens, proteins, nucleic acids, oligonucleotides, peptides, lipids, lectins, inhibitors, activators, ligands, hormones, cytokines, sugars, amino acids, fatty acids, phenols, and alkaloids.

In some embodiments, the one or more sensors are positioned between each of the micropillars. In some embodiments, the one or more sensors are localized surface biosensors. In some embodiments, the device is configured to replicate or mimic a synovium disease or disorder state selected from the group consisting of: RA, osteoarthritis, gout, Lupus, and the like. In some embodiments, a device replicating or mimicking an RA disease state comprises FLS cells in the first outer region.

In some aspects, a method of determining RA treatment responsiveness having the steps of: providing a device mimicking an RA disease state, administering an RA treatment to the central chamber and determining RA treatment responsiveness based on a measured change in the central chamber.

In some embodiments, the RA treatment is a Disease modifying anti-rheumatic Drug (DMARD) selected from the group consisting of: methotrexate, sulfasalazine, leflunomide, hydroxychloroquine and azathioprine. In some embodiments, the measured change is a quantity of live and dead FLS cells after 1-3 days treatment or more. In some embodiments, a culture of RA FLS is on chip and RA patient monocyte perfused through the culture. In some embodiments, a culture of RA FLS is on chip and RA patient monocyte pre-treated with IFN-a and IFN-is perfused through the culture. In some embodiments, a culture of RA FLS is pretreated with IFN-α and IFN-β in different ratios and untreated RA monocyte is perfused through the culture. In some embodiments, a culture of RA FLS is on chip and RA patient monocyte is perfused through the culture followed by a subsequent treatment of the FLS and infiltrating Mo/Mφ with IFNs in different ratios.

### BRIEF DESCRIPTION OF THE DRAWINGS

The following detailed description of exemplary embodiments of the invention will be better understood when read in conjunction with the appended drawings. It should be understood, however, that the invention is not limited to the precise arrangements and instrumentalities of the embodiments shown in the drawings.

FIG. 1A & FIG. 1B depict a birds-eye-view schematic of an exemplary synovium-on-a-chip device according to aspects of the present invention.

FIG. 2A & FIG. 2B depict an enlarged view of the central chamber of an exemplary synovium-on-a-chip device according to an aspect of the present invention.

FIG. 3 is a cross-sectional view of a synovial joint displaying a Normal vs Rheumatoid Arthritic pathomorphology.

FIG. 4A, FIG. 4B, FIG. 4C, FIG. 4D, FIG. 4E, FIG. 4F, FIG. 4G, & FIG. 4H depict an RA-on-chip device and the respective experimental results.

### DETAILED DESCRIPTION

The present invention provides devices that replicate the synovium in a microfluidic chip, and associated methods of use. The devices can be used to model certain disease states related to joint and synovium, such as rheumatoid arthritis remission and relapse under various treatment conditions. The devices can be adapted to replicate synovium from patient-specific cells such that treatment conditions can be modeled and tailored to individual patients. In some embodiments, the devices are suitable for evaluating chemo-and immunotherapies on a patient-specific basis.

### Definitions

It is to be understood that the figures and descriptions of the present invention have been simplified to illustrate elements that are relevant for a clear understanding of the present invention, while eliminating, for the purpose of clarity, many other elements typically found in the art. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Unless defined elsewhere, all technical and scientific terms used herein have the same meaning as commonly understood by one of ordinary skill in the art to which this invention belongs. Although any methods and materials similar or equivalent to those described herein can be used in the practice or testing of the present invention, exemplary methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, and ±0.1% from the specified value, as such variations are appropriate.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 2, 2.7, 3, 4, 5, 5.3, 6, and any whole and partial increments there between. This applies regardless of the breadth of the range.

### Synovium-on-a-Chip

The bioengineered human Synovium-on-a-Chip system allows longitudinal analysis of cells to understand how the synovial environment changes the way cells act during immunomodulation. Manipulation of the chip environments and monitoring the effects by multimodal means provides a new way to interrogate and examine human pathobiology of the synovium. Ultimately, this bioengineered human Synovium-on-a-Chip model can be used as a “clinical trial on a chip” that could pre-screen patients suitable for specific immunotherapies. For example, in certain embodiments, this platform could be used in pre-clinical drug testing to support early-stage drug discovery work for treatments for rheumatoid arthritis (RA) and other disorders of the synovium.

The disclosed 3D microfluidics-based organotypic ‘Synovium-on-a-Chip’ device comprises two distinct functional regions: The inner ring area is cultured with endothelial cells to form a vascular network and the outer ring area is cultured with RA FLS which aligns into synovial layer. The disclosed design provides several methodological features including the capability of control over various biological parameters (e.g. cell type, concentration and composition, tissue architectural information, and extracellular matrix properties), real-time visualization of physiological and pathophysiological dynamics (e.g. cell proliferation and migration, cell fate, and direct and indirect intercellular communications) modulated by internal factors and external stimuli, and the easy setup and compatibility with high throughput on-chip biological assays (e.g. molecular, cellular, and histological characterizations) as well as follow-up cell retrieval for in-depth genetic analyses (e.g. scRNA-seq).

It is to be noted that throughout this disclosure, the terms Synovium-on-a-chip, Rheumatoid Arthritis-on-a-chip, RA-on-a-chip, and Pannus-on-a-chip may be used in reference to the disclosed device.

### Synovium-on-a-chip Device

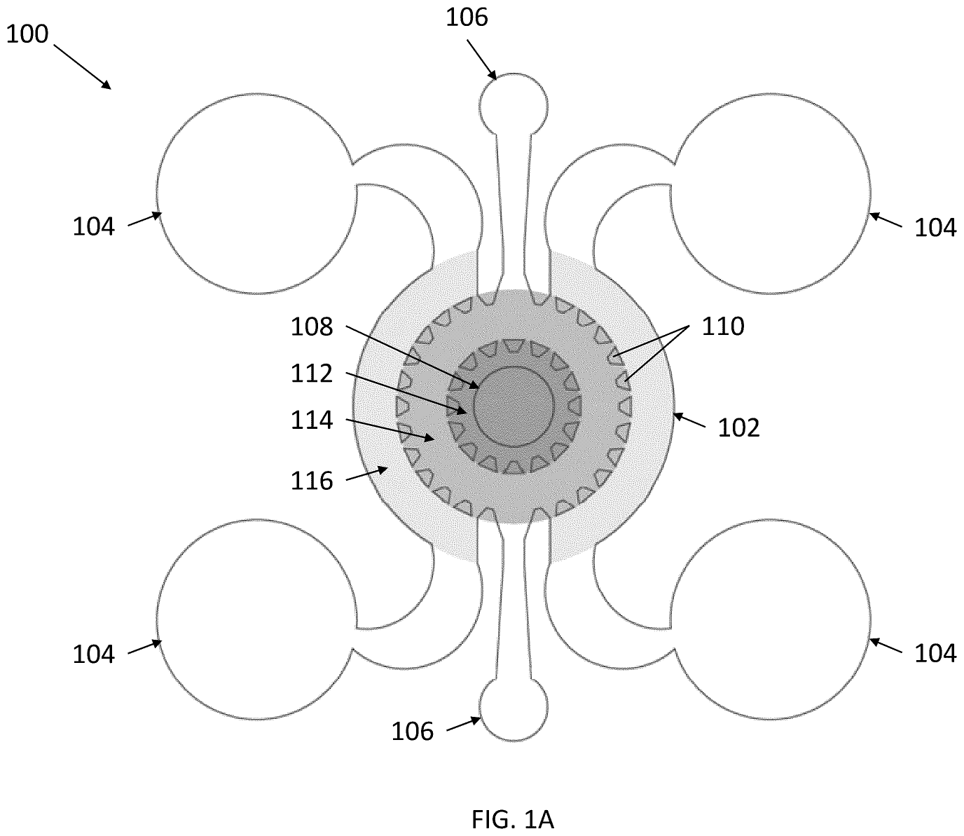

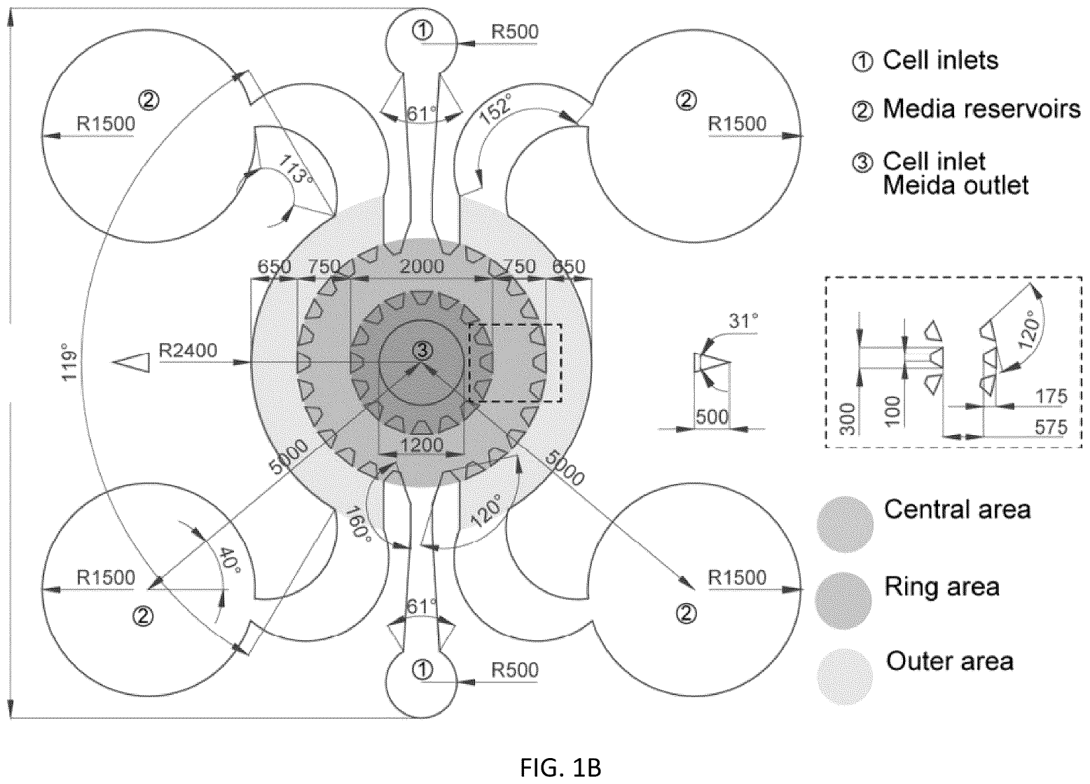

Referring now to FIG. 1A, an exemplary layout of a synovium-on-a-chip device 100 is depicted. Device 100 comprises a cartridge housing containing a central chamber 102 fluidly connected to one or more apertures. For example, central chamber 102 can be fluidly connected to one or more media apertures 104 and one or more cell apertures 106, wherein each fluid connection comprises one or more microchannels. In some embodiments, central chamber 102 can be directly connected to one or more apertures, such as aperture 108. While central chamber 102 is depicted in FIG. 1A as comprising four media apertures 104, two cell apertures 106, and a centrally positioned aperture 108, it should be understood that device 100 can comprise any desired number of apertures in any desired position or arrangement. The housing can be constructed from any desired material and can be at least partially transparent such that central chamber 102 is visible from an exterior of device 100.

Central chamber 102 comprises a substantially circular shape and receives one or more cells for co-culture. Central chamber 102 can be subdivided into a plurality of regions, wherein each region receives a population of cells to mimic native tissue architecture. Central chamber 102 may comprise at least one inner region and at least one outer region. Central chamber 102 may comprise a first and second inner region, and a first and second outer region. Central chamber 102 can be partitioned into each of the regions by a series of micropillars 110. While micropillars 110 are depicted as having a trapezoidal cross-sectional shape, it should be understood that micropillars 110 can have any desired cross-sectional shape, including but not limited to circular, ovoid, square, rectangular, triangular, trapezoidal, polygonal, and the like. In the depicted embodiment, micropillars 110 are regularly-spaced by a distance configured to substantially impede flow of viscous materials such as hydrogel solutions, while permitting flow of liquid materials and diffusion of analytes through capillary action. Such a distance can be between about 50 μm and 200 μm.

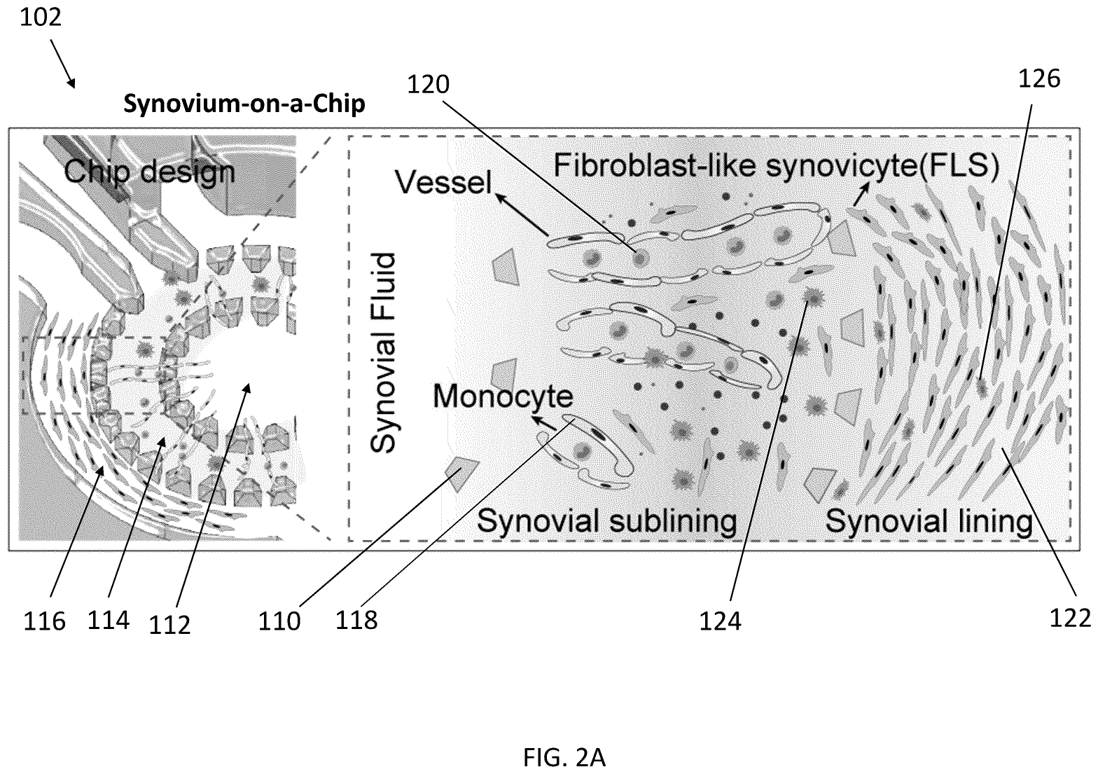

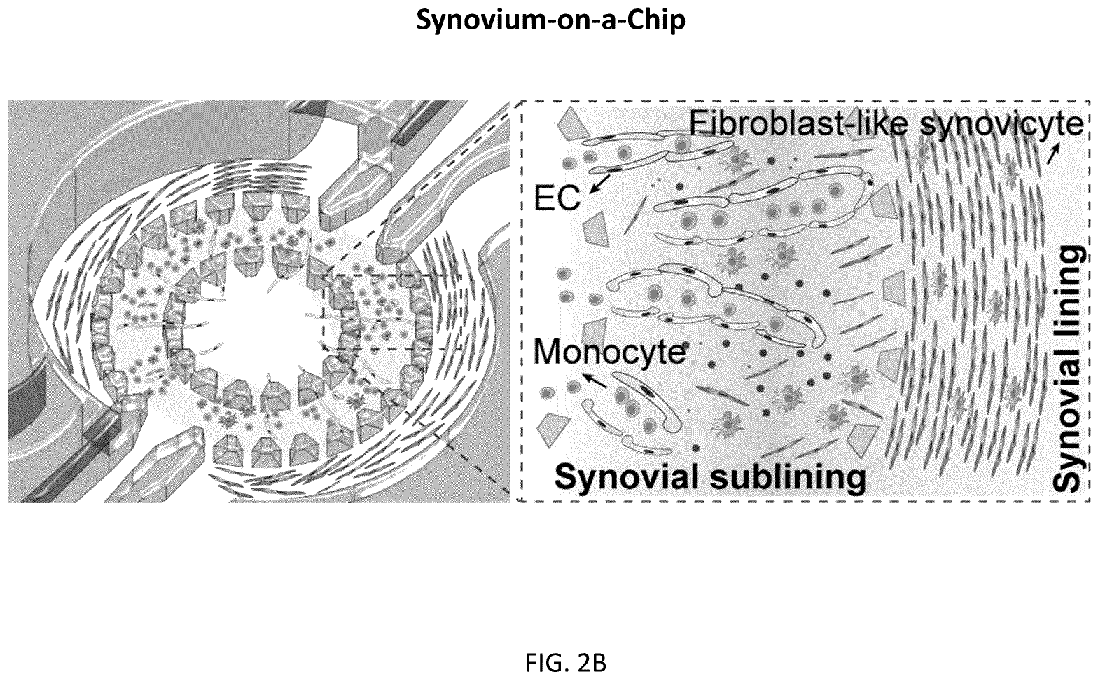

In some embodiments, central chamber 102 can comprise three concentric regions configured to mimic a synovium niche as depicted in FIG. 1A: a central region 112, a middle ring region 114, and an outer ring region 116. Each of the regions can be correspondingly seeded with one or more populations of cells. For example, FIG. 2A depicts an enlarged view of central chamber 102 comprising: central region 112; middle ring region 114 seeded with endothelial cells 118 and outer ring region 116 seeded with FLS 122. Also shown in FIG. 2A are monocytes 120, monocyte-derived macrophages 124, and macrophage-like synoviocytes 126. In some embodiments, outer ring region 116 is seeded with FLS 122 and a Fibrin Solution. In some embodiments, a first inner region is seeded with endothelial cells, and a first outer region is seeded with FLS, and both regions receive Fibrinogen Solution. Other cells that may be introduced or seeded in the various regions of chamber 102 include, but is not limited to, vascular cells, macrophages, macrophage-like synoviocytes, fibroblast-like synoviocytes, Dendritic cells, monocytes, and the like. In certain embodiments, apertures of device 100 are preferentially fluidly connected to a partitioned region. For example, FIG. 1 depicts media apertures 104 being preferentially fluidly connected to outer ring region 116, cell apertures 106 being preferentially fluidly connected to middle ring region 114, and aperture 108 being preferentially fluidly connected to central region 112.

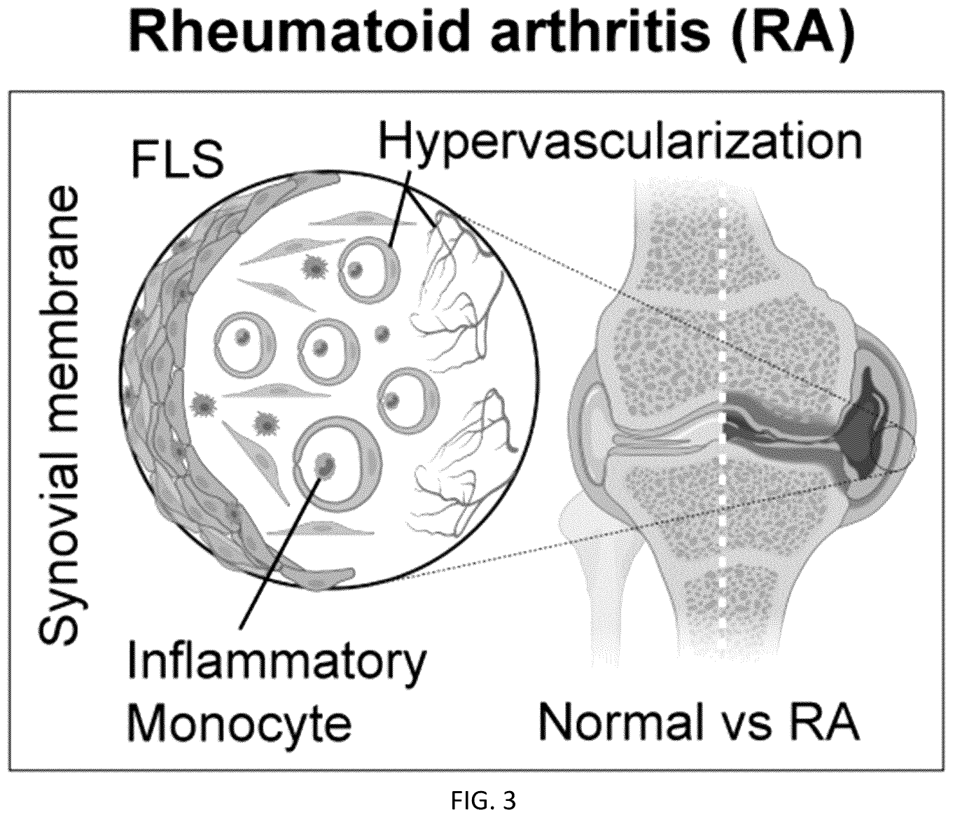

In various embodiments, device 100 can be used to replicate or mimic synovium niche under certain disease or disorder states. Now referring to FIG. 3, shown are some morphological differences between a healthy and normal synovium compared to a synovium with RA. Contemplated disease or disorder states include but are not limited to: RA, osteoarthritis, gout, Lupus, and the like. In such states, device 100 can be used to model the progression of a disease or disorder as well as evaluate therapies to treat a disease or disorder.

Cells may be isolated from a number of sources, including, for example, biopsies from living subjects and whole-organs recovered from cadavers. In some embodiments, the isolated cells are autologous cells obtained from a subject. Autologous cells can be used in device 100 to model progression and therapy on a patient-specific basis. In certain embodiments, the cells may be derived from cultured cell lines. In certain embodiments, the cells seeded into the device are differentiated from stem cells.

Seeding of cells into device 100 may be performed in any desired method. In one embodiment, the cells are embedded in a hydrogel solution and injected into a corresponding region of central chamber 102 by way of the one or more apertures.

Injection of hydrogel solution may be accompanied by the application of a gentle vacuum at an oppositely positioned aperture to encourage infiltration of hydrogel solution into a respective region. Contemplated hydrogel solutions include but are not limited to fibrinogen, collagen, hyaluronic acid, alginate, polyacrylamide, polyethylene glycol, and the like. The hydrogel solution can be cross-linked within central chamber 102 based the material used, such as by photo-cross-linking, thermal-cross-linking, chemical cross-linking, and the like.

### Sensors

In some embodiments, device 100 further comprises one or more sensors for rapid analyte detection. The one or more sensors can comprise any desired sensing mechanism commonly used in art, including but not limited to chemically active regions, electrochemical sensors, immobilized capture molecules, probes, and the like. Contemplated probes or capture agents can be any suitable molecule, including antibodies, antibody fragments, antigens, proteins, nucleic acids, oligonucleotides, peptides, lipids, lectins, inhibitors, activators, ligands, hormones, cytokines, sugars, amino acids, fatty acids, phenols, alkaloids, and the like. The probes or capture agents can be configured to capture any desired molecule, including proteins, amines, peptides, antigens, antibodies, nucleic acids, steroids, eicosanoids, DNA sequences, RNA sequences, bacteria, viruses, and fragments thereof.

### RA-on-a-Chip

As described elsewhere herein, the synovium on a chip device of the present invention can be used to model a variety of disease or disorder states in synovium, such as Rheumatoid Arthritis (RA). Accordingly, the present invention further comprises methods of fabricating RA-on-a-chip devices and methods of characterizing RA treatment using the synovium-on-a-chip devices. In some embodiments, RA synovium niche can be replicated or mimicked by providing synovium cells, including but not limited to: vascular cells, macrophages, macrophage-like synoviocytes, fibroblast-like synoviocytes, Dendritic cells, monocytes, and the like.

In some embodiments, a method of the present invention can include a step of providing synovium cells from a source, wherein the source can be a tissue bank, an autologous source, an allogeneic source, or a xenogeneic source. In some embodiments, a method of the present invention can include a step of modifying the provided synovium cells. In some embodiments, a method of the present invention can include a step of providing device 100 seeded with cells as described elsewhere herein to replicate or mimic a synovium niche, and further seeding middle ring region 114 and outer ring region 116 with one or more cells.

The synovium niche can be used to evaluate the effectiveness of RA therapies, including but not limited to NSAIDS, Steroids, Analgesics, Opioids, JAK inhibitors, Conventional Disease modifying anti-rheumatic Drug (DMARDS), Biological Agents, Targeted Synthetic DMARDs.

Accordingly, in some embodiments a method of the present invention can include a step of applying one or more RA treatments to a synovium-on-a-chip device and a step of characterizing the effect of the one or more RA treatments on synovium cells on the synovium-on-a-chip device.

In some embodiments, device 100 adapted to replicate or mimic synovium niche can be used to evaluate therapeutic responsiveness of DMARD, including but not limited to methotrexate, sulfasalazine, leflunomide, hydroxychloroquine and azathioprine. Therapeutic responsiveness can be evaluated over a period of 1-3 days or more. Therapeutic responsiveness can be rated based on the number or percentage of live and dead synovial cells. Therapeutic responsiveness can be rated based on the number of migrated monocytes into the outer ring region. Therapeutic responsiveness can be rated based on the secretion profile of inflammatory cytokines.

In some embodiments, the RA chip is compartmentalized into three functional regions to mimic the native in vivo tissue architecture of synovitis. The different tissue regions are a central sinus in central region 112 that is lined with human umbilical vein endothelial cells (HUVECs), a synovial sublining, which contains vessels extending from the central sinus, and a synovial lining region (outer ring region 116) connected with four medium reservoirs for medium supply. HUVECs and RA patient derived FLS, and RA patient monocyte-derived macrophage are infused into different regions sequentially and cultured for 7 days to establish a microvessel network and a layered structure of synovium.

### Fabrication

The synovium on a chip device of the present invention is made using any suitable method known in the art. The method of making varies depending on the materials used. For example, in some embodiments, components substantially comprising a metal are milled from a larger block of metal or are cast from molten metal. Likewise, in some embodiments, components substantially comprising a plastic or polymer are milled from a larger block, cast, or injection molded. In some embodiments, the components are made using 3D printing or other additive manufacturing techniques commonly used in the art. In some embodiments, microstructures and patterns are achieved through microfabrication techniques including but not limited to: lithography, thin film deposition, electroplating, etching, micromachining, and the like.

### Synovium on a Chip Kits

The present invention also provides kits for replicating or mimicking synovium niche. The kits include the synovium on a chip and RA on a chip devices described elsewhere herein, as well as relevant materials, reagents and instrumentation. For example, in some embodiments, the kit can comprise reagents for loading and culturing cell populations, including but not limited to hydrogels for 3D cell culture, cell culture media, wash media, and the like. In some embodiments, the kit can comprise instrumentation for manipulating contents of the synovium/RA on a chip devices, including but not limited to pipettes, pipette tips, syringes, and the like. In some embodiments, the kit can comprise one or more capture molecules or probes as described elsewhere herein, wherein a user can select the one or more capture molecules or probes for inclusion in the sensors of the synovium/RA on a chip devices to detect and/or quantify one or more analytes of interest.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art may, using the preceding description and the following illustrative examples, utilize the present invention and practice the claimed methods. The following working examples therefore, specifically point out exemplary embodiments of the present invention, and are not to be construed as limiting in any way the remainder of the disclosure.

### Example 1: Background & Applications

Inadequately treated rheumatoid arthritis (RA) results in increased morbidity and mortality, and heavy economic burden associated with loss of function.

Unfortunately, current treatment strategies are trial-and-error because there are no markers available to determine which therapy is best for an individual, and many patients cycle through ineffective therapies and accrue damage. Tumor necrosis factor-alpha inhibitors (TNFi) are the most common biologic treatment in RA, yet responses vary, with 30% not responding and 30% responding partially.

To study blood and synovial cells from RA patients to understand the biological basis for response/non-response to TNFi, disclosed herein is a microfluidics-based 3D vascularized organ-on-a-chip model of RA. The disclosed microphysiological system integrates key features that replicate in vivo synovial architecture and monitors dynamic interactions between the critical cell types of RA, such as monocyte, monocyte-derived macrophage, and fibroblast-like synoviocytes (FLS), in real time with live-cell imaging. The RA chip is compartmentalized into three functional regions, which are partitioned by regularly spaced trapezoid micropillars that confine cell-embedded hydrogels, by balancing surface tension and capillary forces, to mimic the native in vivo tissue architecture of synovitis. The different tissue regions are a central sinus that is lined with human umbilical vein endothelial cells (HUVECs), a synovial sublining, which contains vessels extending from the central sinus, and a synovial lining region (outer ring) connected with four medium reservoirs for medium supply. HUVECs and RA patient derived FLS, and RA patient monocyte-derived macrophage are infused into different regions sequentially and cultured for 7 days to establish a microvessel network and a layered structure of synovium.

With this established synovium structure on-chip, first profiled is the cytokine environment of the engineered RA chip at two conditions (with TNF-α priming versus without) to validate the inflammation status of the RA chip. It is established that TNF-α priming significantly induced an inflammatory environment within the RA chip with increased secretion of a myriad of cytokines, such as IL-6, GM-CSF, IL-8, MCP-1, MCP-3, and MIP-3α. Additionally, different setups of RA chip with single or companion infusion of macrophage and FLS showed different extent of inflammatory response in term of IL-6 secretion, where co-presence of macrophage and FLS demonstrated the highest response to TNF-α priming, as compared to infusion of macrophage and FLS separately, highlighting the interaction between macrophage and FLS may be involved in regulation of RA inflammation.

Following the established inflammation in the RA chip, work pivoted to whether the RA inflammation can be maintained on-chip for a long period. The RA chip cultured for over 4 days and quantified inflammatory cytokines secretion with IL-6 and GM-CSF as indicator. Unexpectedly, it was found that secretion of IL-6 and GM-CSF generally decreased, highlighting the discontinuation of inflammation status of RA chip primed by transient TNF-α treatment. Clinical observation of RA patients demonstrated inflammation flareups with ups and downs. To mimic these scenarios, the RA chip is primed with TNF-α at an interval of 2-3 days and the resultant inflammatory environment was found to be maintained over 7 days. It was previously shown that pre-treatment circulating type I IFN (T1IFN) activity predicts non-response to TNFi (Wampler-Muskardin et al., Front Immunol, 2020). Remarkably, no patient with a ratio >1.3 achieved remission or low disease activity. It is also possible to use single cell gene expression analysis to study blood monocytes in RA patients with high vs. low IFN-β-to-α activity ratio. These data support a major downstream effect of the T1IFN ratio upon a critical effector cell population, monocyte. Further on, the behaviors of monocytes in the RA chip are investigated by infusing monocytes into the central sinus of the chip, from which monocytes can distribute into the microvessel network and extravasate out of vessel. It was discovered that inflammatory RA niche promoted monocyte migration dynamics in terms of migration velocity and distance, as compared to that not primed by TNF-α. In addition, the effect of circulating T1IFNs on monocyte activation in the RA chip was studied. It was discovered that IFN-β priming may suppress CD16 expression on monocytes.

Currently, the relative contributions of blood vs. synovial signals in the diapedesis and differentiation of monocytes to inflammatory monocyte/macrophages remains not known. It is hypothesized that increased IFN-β signaling in monocyte alters its diapedesis and that IFN-stimulated FLS promotes a pro-inflammatory macrophage phenotype. The study of monocytes expands with samples collected from RA patients and donor RA FLS. Participants are biopsied prior to biologic immunosuppressive therapy. Clinical data for each subject, including demographics, smoking status, BMI, and disease activity measures are collected. Blood is collected on the same date as the synovial biopsy. The data collected using patient samples provides key preliminary data for an NIH R series grant focusing on patient-specific RA chip to be submitted in 2022.

### Example 2: Synovium-on-a-chip Device Construction

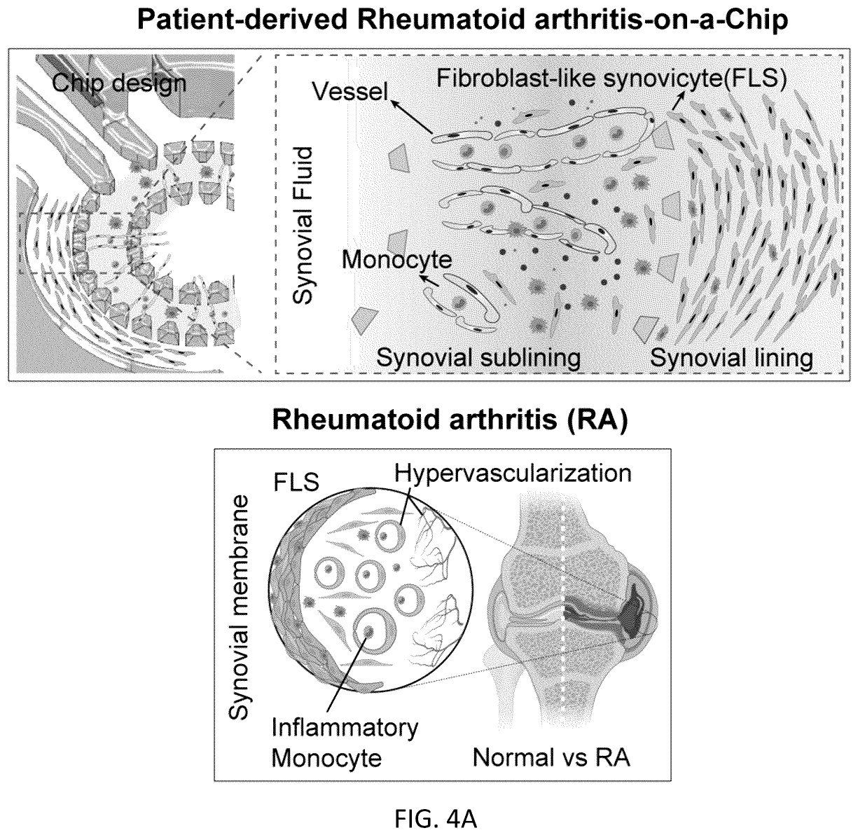

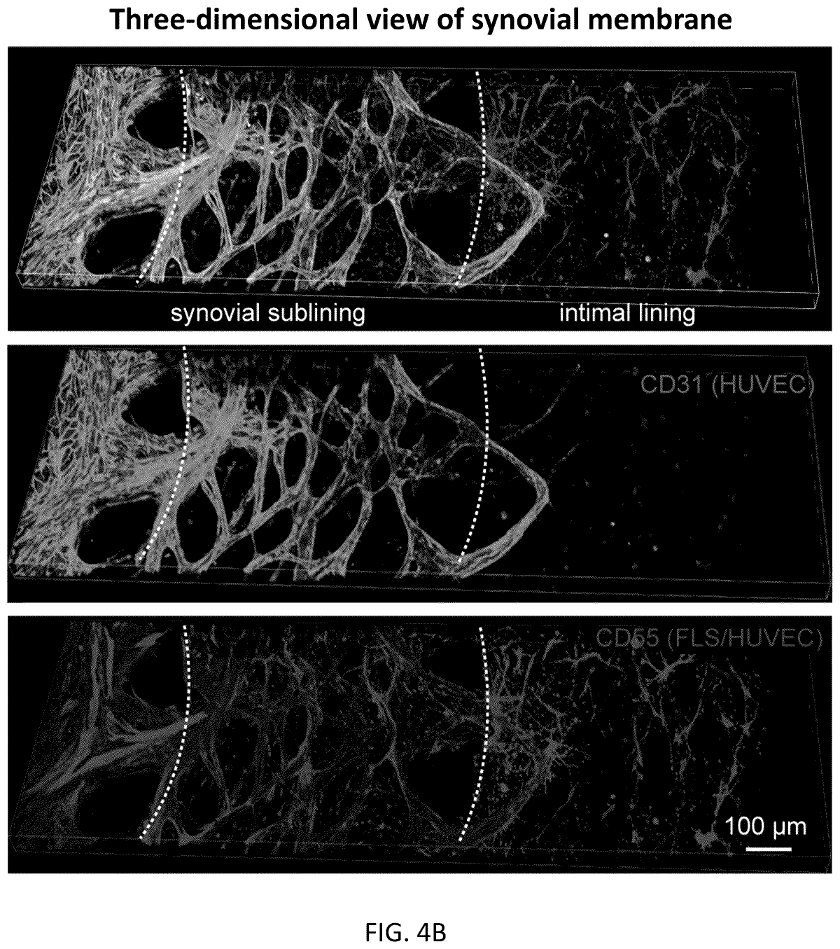

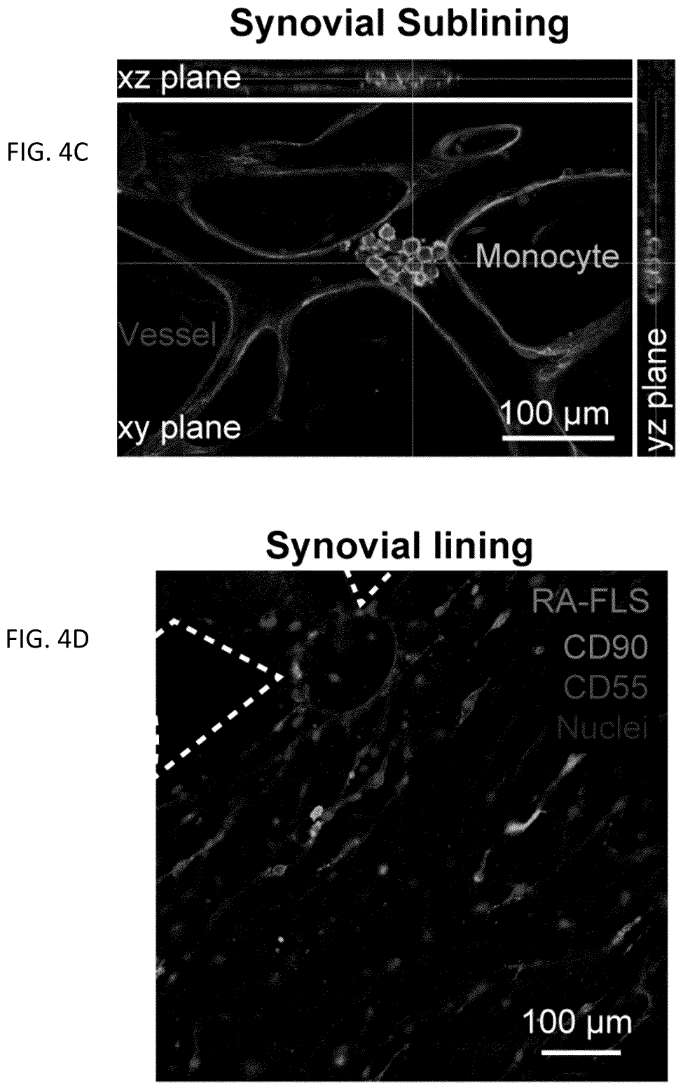

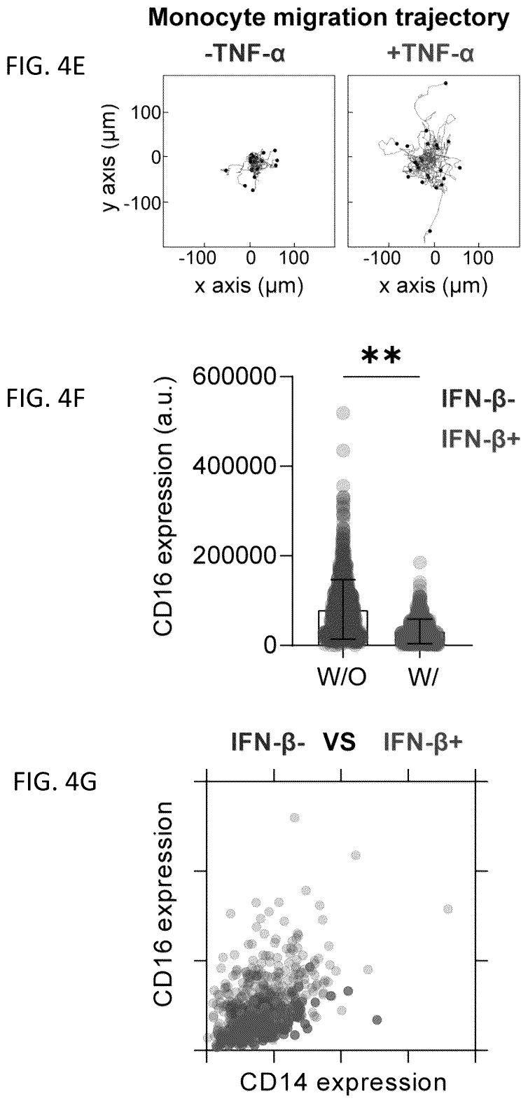

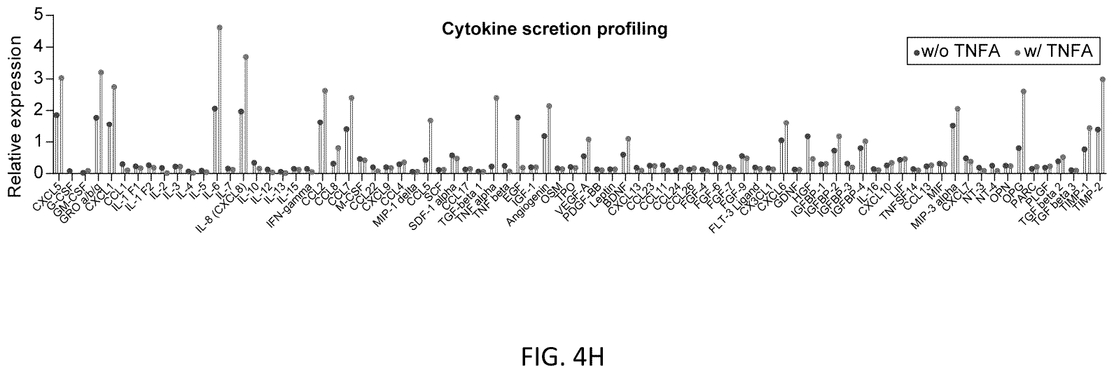

The biomimetic Synovium-on-a-Chip (FIG. 4A) contains two areas to mimic the outer layer (lining layer) and an inner layer (sublining layer) of the synovium. The inner ring area was infused with endothelial cells to form a vascular network and the outer ring area was inoculated with RA FLS which aligned into synovial layer. The resultant system consists of a vascularized sublining layer (CD31 staining, green) which is surrounded by the synovial lining layer (CD55 staining, red), mimicking the synovium structure in vivo (FIG. 4B, FIG. 4C, & FIG. 4D). To study the interaction between monocyte and RA FLS in the RA niche, monocytes are loaded (yellow) into the vasculature and migration behaviors in response to RA milieu is studied with live imaging microscopy (FIG. 4C).

To confirm the role of TNF-α in regulating the RA inflammation, monitored first is monocyte dynamics in the RA device which is primed with or without TNF-α. Enhanced migration behaviors of monocytes were discovered in the inflammatory environment (FIG. 4E). Previously, the biology of non-classical and classical monocytes was examined from RA patients defined by their pre-biologic treatment T1IFN activity. It was found that low IFN-β-to-α activity ratio but not high ones strongly aligned with JAK1 expression in monocyte and mostly importantly, high IFN-β-to-α activity (ratio>1.3) correlated with no response in RA patients with iTNF treatment (Witkowski, Matthew T., et al. “Extensive remodeling of the immune microenvironment in B cell acute lymphoblastic leukemia.” Cancer cell 37.6 (2020): 867-882). Following clinical observation, the monocyte phenotype was profiled and it was found that IFN-β primed CD14+ monocytes, as compared to those without IFN-β priming, demonstrated lower expression of CD16 (FIG. 4F & FIG. 4G).

To further understand the underlying mechanisms, the cytokine secretion profile of the RA niche was mapped (FIG. 4H). The results identified several cytokine factors, such as IL-6, CCL5, and CCL8, which may be involved in recruiting monocyte into the RA niche, thus driving the inflammation.

### Example 3: IFN-α and IFN-β on RA Monocytes

Goal: determine the impact of IFN-α and IFN-β on RA monocytes (Mo), Mo-derived macrophages (Mφ), and fibroblast-like synoviocytes (FLS).

It is hypothesized that increased IFN-β relative to IFN-α results in increased Mo diapedesis and promotes a pro-inflammatory Mφ phenotype. This hypothesis is tested using a novel vascularized pannus-on-a-chip model, allowing the study of diapedesis and interactions with human synovial cell types in vitro. Mo pretreated +/−IFN-β or IFN-α with FLS cells in the chip +/−IFN-β or IFN-α in various combinations are compared to definitively assess the impact of T1IFN on circulating cells vs. synovial cell types on diapedesis and inflammatory Mo differentiation in tissue. Cellular architecture, gene expression, and cytokine secretion are analyzed. Flow cytometry is used to assess cells after culture, including inflammatory activation markers as well as osteoclast precursor markers (c-Fms and RANK) on Mo and Mφ and Thy-1 on FLS. This data correlates the effects of IFN pre-treatment on infiltrating Mo vs. FLS and assesses the relative contributions of IFNs on the stromal cells vs. infiltrating immune cells. Results are compared with standard co-culture and trans-well experiments of the same cell types and conditions to determine the contributions of diapedesis and soluble vs. cell contact factors.

### Example 4: Patients and Samples

Monocytes are studied from RA patients and donor RA FLS. Participants are biopsied prior to biologic immunosuppressive therapy. Clinical data for each subject, including demographics, smoking status, BMI, and disease activity measures are collected. Blood is collected on the same date as the synovial biopsy. Synovial biopsy samples are obtained under ultrasound guidance using needle biopsy.

### Example 5: Pannus-on-a-Chip

A novel, microphysiological, and perfusable (vascularized) organ-on-a-chip model is utilized to understand the contribution of type I IFN to inflammation and treatment response in RA. Monocultures of Mo, Mφ, and FLS are effective for transcript analyses and understanding individual contributions to the RA joint environment but omit an important part of RA biology which depends on contextual environment and pannus architecture. Simulating the structure of the synovium with a biomimetic co-culture system provides a way to observe the interactions between the cell types in a tissue context in vitro as they are exposed to controlled RA-like environments. This bioengineered organ-on-chip device was recently developed by Weiqiang Chen (Wampler Muskardin, Theresa L., et al. “Distinct single cell gene expression in peripheral blood monocytes correlates with tumor necrosis factor inhibitor treatment response groups defined by type i interferon in rheumatoid arthritis.” Frontiers in immunology 11 (2020): 1384; Ma, Chao, et al. “Organ-on-a-chip: a new paradigm for drug development.” Trends in pharmacological sciences 42.2 (2021): 119-133). The device is adapted to achieve a perfusable pannus-on-a-chip model, which is ideal for examining effects of T1IFNs on infiltrating and stromal cells, and which can be further adapted to include additional synovial cell types and to test additional cytokines or therapies in the future.

The disclosed microfluidics-based microphysiological system integrates key features that replicate in vivo synovial architecture and monitors dynamic Mo, Mo-derived Mφ, and FLS interactions in real time with live-cell imaging. The pannus-on-a-chip culture is compartmentalized into a central sinus that is lined with human umbilical vein endothelial cells (HUVEC/ECs); a synovial sublining, which contains vessels extending from the central sinus; and synovial lining region (outer ring) connected with four medium reservoirs for medium supply. These functional regions are partitioned by regularly spaced trapezoid micropillars that confine cell-embedded hydrogels, by balancing surface tension and capillary forces, to mimic the native in vivo tissue architecture of synovitis. This system is designed to create synovial sublining and lining regions (FIG. 2B). Pannus-on-a-chip experiments include: (i) Culture of RA FLS on chip with RA patient Mo perfused through the culture. (ii) Culture of RA FLS on chip with RA patient Mo pre-treated with IFN-α and IFN-β in various ratios prior to perfusion through the culture. (iii) Pretreatment of RA FLS with IFN-α and IFN-β in different ratios with untreated RA Mo perfused through the culture. (iv) Culture of RA FLS on chip with RA patient Mo perfused through the culture and subsequent treatment of the FLS and infiltrating Mo/Mφ with IFNs in different ratios. General protocol: RA FLS and HUVEC are loaded into devices and cultured for 7 days. Half are treated with T1IFN-spiked medium on day 6. CD14+ Mo from PBMC are divided into 2 groups. One is primed with T1IFN for 2 days before being loaded onto the device on day 7 of FLS and HUVEC cultures. Staining and photographing occur on day 10. In parallel, the effects on the cells in monoculture as well as co-culture in transwell plates are examined.

Co-cultures include: 1) Mo pre-treated with IFN-α and IFN-β in different ratios and then applied to FLS, 2) untreated Mo applied to FLS pre-treated with IFN-α and IFN-β in different ratios, and 3) untreated Mo and FLS cultured together and treated simultaneously with IFN-α and IFN-β in different ratios. These conditions are compared to each other, to control cultures with no IFN stimulation, and to the results of the organ-on-a-chip experiments. Response to exposure to various combinations of IFN-α and IFN-β for 6, 18, and 24H are assessed.

For all cultures, cellular phenotype, gene expression, and cytokine secretion are analyzed. Flow cytometry is used to assess Mo, Mφ and FLS cells, including markers of inflammatory activation (IL1B); IFN pathway activation (IFI6); osteoclast precursor markers (c-Fms and RANK); CD11b, CD68, CD16, CD14 on Mo and Mφ; and THY1, HLA-DR, DKK3, CD34, and cadherin-11 on FLS (Zhang, Fan, et al. “Defining inflammatory cell states in rheumatoid arthritis joint synovial tissues by integrating single-cell transcriptomics and mass cytometry.” Nature immunology 20.7 (2019): 928-942). Cytokines will be measured in supernatants. Cells isolated following culture are purified into Mo/Mφ and FLS lineages. Gene expression are measured in these cell types using RNA-seq, and transcripts of exceptional interest will be specifically examined by qPCR. Phenotypic markers are quantified and coupled with spatial analyses to map cell interactions and invasiveness. Results are compared between the culture conditions to determine the impact of different type I IFNs upon Mo, Mo-derived Mφ and FLS cells.

### Example 6: Relevance to Autoimmunity

The bioengineered human organ-on-a-chip model is an alternative to animal models. Manipulation of the chip environments and monitoring the effects by multimodal means provides a new way to interrogate and examine human pathobiology. Once optimized, the disclosed pannus-on-a-chip model has the potential to achieve a so-called “clinical trial on a chip” that could pre-screen patients suitable for specific immunotherapies. This platform could be used in pre-clinical drug testing in RA to support early stage drug discovery work.

### Example 7: Design & Fabrication

FIG. 1B depicts the design and fabrication of the biomimetic Synovium-on-a-Chip platform. It is to be noted the unit in FIG. 1B is μm. FIG. 1B depicts an exemplary schematic demonstrating the fabrication process of the devices using photolithography and soft lithography [Ma, Chao, et al. “On-chip construction of liver lobule-like microtissue and its application for adverse drug reaction assay.” Analytical chemistry 88.3 (2016): 1719-1727]. The device mold is fabricated by patterning photoresist onto a silicon wafer using a high-resolution mask printed from (FIG. 1B). Uncured PDMS is cast onto a silicon master mold and cured at 80° C. for 1 hr to produce a thick PDMS layer, punched with holes and bonded to cover glass for framing 3D hydrogel. The cell loading process is now discussed. First, the sacrificial gelatin hydrogel solution (12 mg/ml in DPBS) is injected 10 into the central area and solidified at 4° C. for 15 min. Then, a mixture of HUVECs and fibrin solution (3 mg/ml in DPBS) containing 4 U/ml thrombin is infused into in the ring area and gelled at room temperature for 10 min.

To comparatively study the lining and sublining heterogeneity of synovium niche, a mixture of RA-FLS and fibrin solution (3 mg/ml in DPBS) containing 4 U/ml thrombin is loaded into the outer area by a gentle vacuum suction. Following the gelation, cell culture media is added into the four media reservoirs and the device is incubated at 37° C. for 10 min. The gelled gelatin then becomes liquefied and is removed thereafter. At last, HUVECs are seeded at the central area. The established devices are cultured and monitored during the 7-day experiment protocol. The protocol involves the whole scanning of the synovium structure. The resultant system consists of a vascularized sublining layer (CD31 staining, green) which is surrounded by the synovial lining layer (CD55 staining, red), mimicking the synovium structure in vivo (FIG. 4B, FIG. 4C, & FIG. 4D). To study the interaction between monocyte and RA FLS in the RA niche, monocytes (shown in yellow) are loaded into the vasculature from aperture 108 and studied its migration behaviors in response to RA milieu with live imaging microscopy (FIG. 4C).

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A synovium on a chip device, comprising:
a cartridge housing;
a central chamber embedded in the cartridge housing;
at least one aperture fluidly connected to the central chamber; and
a plurality of evenly spaced micropillars arranged in a substantially circular shape within the central chamber such that the central chamber is partitioned into at least a first inner region and a first outer region;
wherein the first inner region comprises endothelial cells configured to mimic a vascular network and the first outer region comprises fibroblast-like synoviocytes (FLS) configured to mimic a synovium.

2. The device of claim 1, wherein the first inner region and the first outer region are concentric.

3. The device of claim 1, wherein the first outer region comprises a fibrin hydrogel where fibroblast-like synoviocytes (FLS) are embedded to mimic a synovium.

4. The device of claim 1, wherein the plurality of micropillars have a cross-sectional shape selected from the group consisting of: circular, ovoid, square, rectangular, triangular, trapezoidal, and polygonal.

5. The device of claim 1, wherein the plurality of micropillars is evenly spaced by a distance between about 50 μm and 200 μm.

6. The device of claim 1, further comprising one or more sensors comprising capture molecules or probes positioned within the central chamber.

7. The device of claim 6, wherein the capture molecule or probe is selected from the group consisting of: antibodies, antibody fragments, antigens, proteins, nucleic acids, oligonucleotides, peptides, lipids, lectins, inhibitors, activators, ligands, hormones, cytokines, sugars, amino acids, fatty acids, phenols, and alkaloids.

8. The device of claim 6, wherein the one or more sensors are positioned between each of the micropillars.

9. The device of claim 6, wherein the one or more sensors are localized surface biosensors.

10. The device of claim 1, wherein the device is configured to replicate or mimic a synovium disease or disorder state selected from the group consisting of: RA, osteoarthritis, gout, Lupus, and the like.

11. The device of claim 10, wherein a device replicating or mimicking an RA disease state comprises FLS cells in the first outer region.

12. A method of determining RA treatment responsiveness, comprising the steps of:
providing the device of claim 11;
administering an RA treatment to the central chamber; and
determining RA treatment responsiveness based on a measured change in the central chamber.

13. The method of claim 12, wherein the RA treatment is a Disease modifying anti-rheumatic Drug (DMARD) selected from the group consisting of: methotrexate, sulfasalazine, leflunomide, hydroxychloroquine and azathioprine.

14. The method of claim 13, wherein the measured change is a quantity of live and dead FLS cells after 1-3 days treatment or more.

15. The method of claim 13, wherein a culture of RA FLS is on chip and RA patient monocyte perfused through the culture.

16. The method of claim 13, wherein a culture of RA FLS is on chip and RA patient monocyte pre-treated with IFN-α and IFN-β is perfused through the culture.

17. The method of claim 13, wherein a culture of RA FLS is pretreated with IFN-α and IFN-β in different ratios and untreated RA monocyte is perfused through the culture.

18. The method of claim 1, wherein a culture of RA FLS is on chip and RA patient monocyte is perfused through the culture followed by a subsequent treatment of the FLS and infiltrating Mo/Mφ with IFNs in different ratios.
